(19) United States
(12) Patent Application Publication
(10) Pub. No.: US 2023/0274803 A1
(43) Pub. Date: Aug. 31, 2023

(54) MOLECULAR DESCRIPTORS BASED ON DISTANCE BETWEEN SUBSTRUCTURES

(71) Applicant: International Business Machines Corporation, Armonk, NY (US)

(72) Inventors: LISA UEKI, Tokyo-to (JP); Seiji Takeda, Tokyo (JP)

(21) Appl. No.: 17/652,796

(22) Filed: Feb. 28, 2022

(51) Int. Cl. G16C 20/30

(52) U.S. Cl. CPC G16C 20/30

(57) ABSTRACT

A method, system, and computer program product are disclosed. The method includes receiving a target molecule and extracting substructures from the target molecule. The method also includes generating a distance matrix for a pair of the substructures based on corresponding atom indexes and determining distances between atoms of the pair based on the distance matrix. Further, the method includes calculating a distance between the pair of the substructures based on at least one of the distances and generating a molecular descriptor based on the distance.

### BACKGROUND

The present disclosure relates to materials analytics and, more specifically, to generating molecular descriptors based on global structure information.

Machine-learning techniques can be used to generate novel molecules for optimization of desired chemical and physical properties. Large numbers of novel molecules that satisfy certain constraints on chemical/physical properties can be generated based on predictive models. The predictive models can be built based on molecular descriptors, which are features of chemical structures. Generating new molecular structures predicted to have desired properties can be used to develop new pharmaceutical compounds, compounds for organic light emitting diodes (OLEDs), polymers, etc.

### SUMMARY

Various embodiments are directed to a method, which includes receiving a target molecule and extracting substructures from the target molecule. The method also includes generating a distance matrix for a pair of the substructures based on corresponding atom indexes and determining distances between atoms of the pair based on the distance matrix. Further, the method includes calculating a distance between the pair of the substructures based on at least one of the distances and generating a molecular descriptor based on the distance.

Further embodiments are directed to a system, which includes a memory and a processor communicatively coupled to the memory, wherein the processor is configured to perform the method. Additional embodiments are directed to a computer program product, which includes a computer readable storage medium having program instructions embodied therewith, the program instructions executable by a processor to cause a device to perform the method.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 is a block diagram illustrating a molecular descriptor generating environment, according to some embodiments of the present disclosure.

FIG. 2A is a schematic diagram illustrating an example wherein substructures are generated for a target molecule, according to some embodiments of the present disclosure.

FIG. 2B is a schematic diagram illustrating an example wherein atom indexes are retrieved for the substructures extracted in the previous example, according to some embodiments of the present disclosure.

FIG. 2C is a schematic diagram illustrating an example wherein topological distances are determined for the substructures, according to some embodiments of the previous disclosure.

FIG. 3 is a flow diagram illustrating a process of selecting identifier definitions from a document, according to some embodiments of the present disclosure.

FIG. 4 is a block diagram illustrating a computer system, according to some embodiments of the present disclosure.

FIG. 5 is a block diagram illustrating a cloud computing environment, according to some embodiments of the present disclosure.

FIG. 6 is a block diagram illustrating a set of functional abstraction model layers provided by the cloud computing environment, according to some embodiments of the present disclosure.

### DETAILED DESCRIPTION

Aspects of the present disclosure relate generally to the field of computational molecular design, and in particular to modeling chemical structures based on molecular descriptors. While the present disclosure is not necessarily limited to such applications, various aspects of the disclosure may be appreciated through a discussion of various examples using this context.

Generating new molecular structures predicted to have desired properties can be used to develop new pharmaceutical compounds, compounds for organic light emitting diodes (OLEDs), polymers, etc. Machine-learning techniques can be used to generate novel molecules for optimization of desired chemical and physical properties. Large numbers of novel molecules that satisfy certain constraints on chemical/physical properties can be generated based on predictive models. The predictive models can be built based on molecular descriptors, which are features of chemical structures. For example, models can use similarity search techniques that characterize atoms by element type and/or count small substructures. However, these approaches do not consider molecules' macroscopic or global structural patterns and are thus insufficient for large-scale material discovery.

Chemical properties, particularly those of organic molecules, strongly depend on intermolecular interactions such as molecular orbitals, electron repulsion, etc. These features are affected by positional relationships between substituents and are important to consider when predicting properties. Examples of molecular descriptors based on positional relationships can include atom pairs fingerprints and topological torsion. However, these capture only atom and bond types, whereas intermolecular interactions typically occur between sets of molecular substructures rather than atoms themselves.

Disclosed herein are techniques for generating molecular descriptors based on topological distances between molecular substructures (e.g., heteroatoms, fragments, and small entities). These descriptors may lead to predictive models with greater accuracy than those based on atom-atom interactions. Further, the disclosed molecular descriptors can be advantageous for designing large molecular structures (e.g., organic light-emitting diode (OLED)-related materials), which can have properties strongly dependent on macroscale features and interactions.

It is to be understood that the aforementioned advantages are example advantages and should not be construed as limiting. Embodiments of the present disclosure can contain all, some, or none of the aforementioned advantages while remaining within the spirit and scope of the present disclosure.

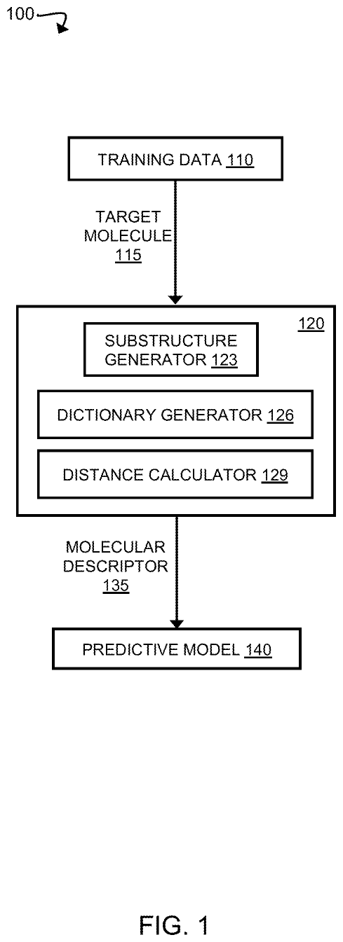

Turning now to the figures, FIG. 1 is a block diagram illustrating a molecular structure predictive modeling environment 100, according to some embodiments of the present disclosure. Environment 100 includes a target molecule 115 that can be from a set of training data 110. The target molecule 115 is a machine-readable representation of a molecule, such as an organic or organometallic small molecule, oligomer, polymer, etc. Examples of machine-readable molecular representation formats can include line notation-based encoding, such as simplified molecular-input line-entry system (SMILES). SMILES can use character strings, lists of tokens, parse trees, and/or molecular graphs to represent valence bond models. SMILES can also specify features such as atoms, bonds, rings, branching, disconnections, isomerism, and reactivity. While molecules represented by SMILES character sequences are illustrated herein, other molecular representations may be used in some embodiments (e.g., International Union of Pure and Applied Chemistry (IUPAC) chemical identifier (InChI) formats, molecular graphs, or any appropriate structural representation).

The target molecule 115 can be input into a molecular descriptor generator 120, which includes a substructure generator 123, a dictionary generator 126, and a distance calculator 129. When the target molecule 115 is received, the substructure generator 123 can extract substructures from the target molecule 115. The substructures can include heteroatoms (i), fragments (ii), and small entities (iii). In some embodiments, the fragments (ii) can be found using BRICS (Breaking of Retrosynthetically Interesting Chemical Substructures), and the small entities (iii) can be found based on the molecular fingerprint, such as a Morgan fingerprint. Herein, a “small entity” can include three or more non-hydrogen atoms. In some embodiments, small entities have three or more carbon atoms.

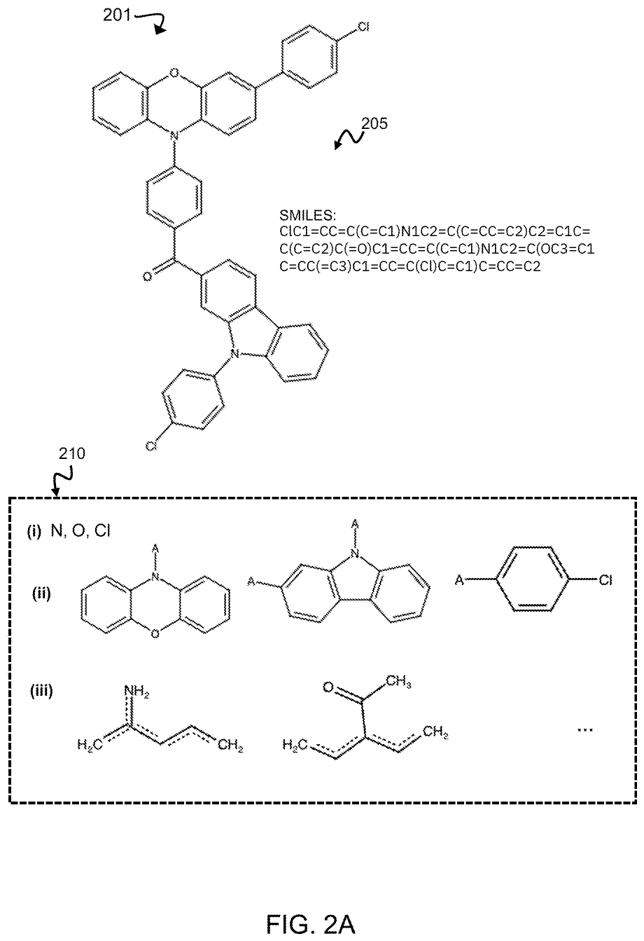

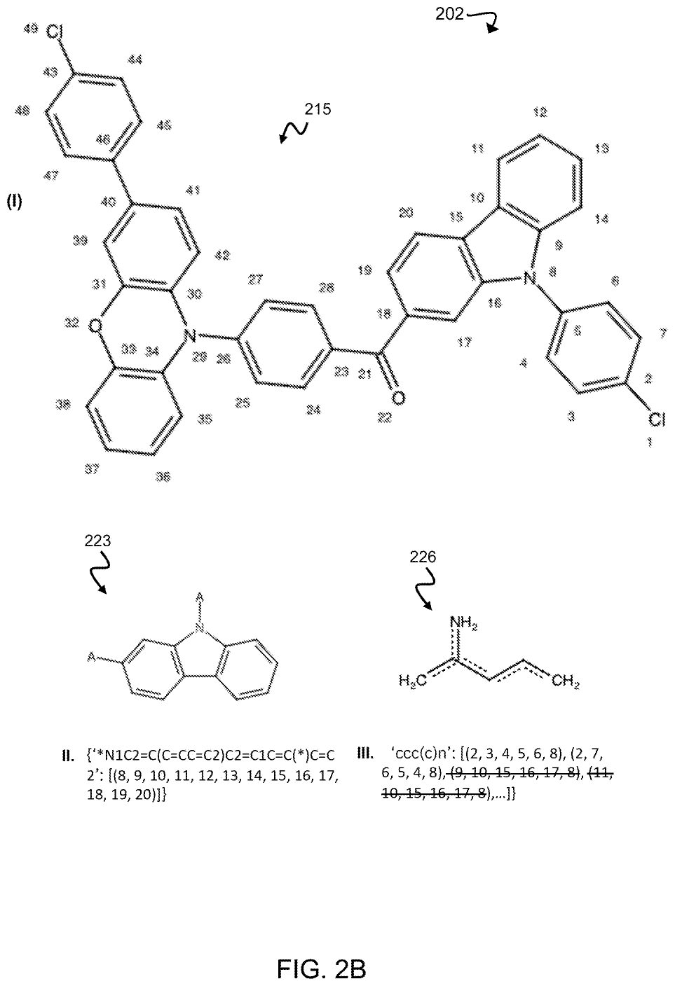

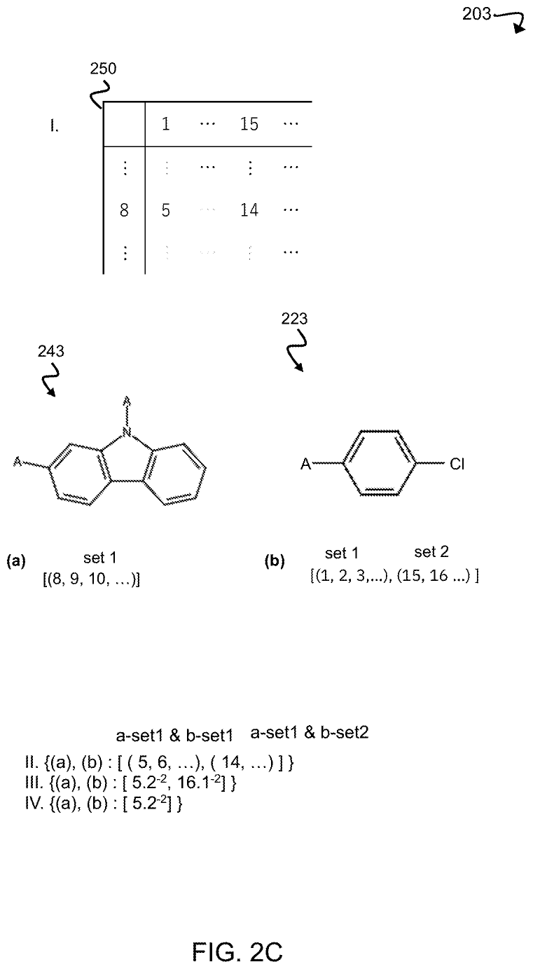

FIG. 2A is a schematic diagram illustrating an example 201 wherein substructures (i), (ii), and (iii) are generated based on a SMILES representation 205 of a target molecule (e.g., target molecule 115), according to some embodiments of the present disclosure. The substructures (i), (ii), and (iii) 210 can be extracted by the substructure generator 123 and include nitrogen (N), oxygen (O), and chlorine (Cl) heteroatoms (i). The substructures 210 also include cleaved aromatic ring fragments (ii), wherein A is a “dummy” atom representing each cleavage site, and small entities (iii) that each contain at least one sequence of three carbon atoms.

Referring again to FIG. 1, the dictionary generator 126 retrieves sets of (non-hydrogen) atom indexes matching the extracted substructures (i), (ii), and (iii) (e.g., substructures 210). The atom indexes may be retrieved in the order (i), (ii), (iii). When retrieving (iii), small entities with larger molecular masses can be searched first. The dictionary generator 126 generates entries for each substructure and corresponding atom index numbers. In some embodiments, sets of atom indexes for small entities that are completely contained within another substructure (e.g., a larger small entity) can be eliminated in order to avoid duplication. For example, atom indexes can be compared in order to eliminate the smaller entities contained within larger entities.

FIG. 2B is a schematic diagram illustrating an example 202 wherein atom indexes are retrieved for the substructures 210 extracted in the previous example 201, according to some embodiments of the present disclosure. In this example 202, a labeled structure 215 of the molecule 205 shown in FIG. 2A is illustrated with atom index numbers [1]-[49]. A pair of substructures including a fragment 223 and a small entity 226 from the extracted substructures 210 are illustrated. The dictionary generator 126 can add atom index numbers (e.g., non-hydrogen atoms) for the fragment 223 and the small entity 226 to respective dictionary entries. The dictionary entry for the small entity 226 can include index numbers for each small entity having this structure in the molecule 215. By comparing the entries for the fragment 223 and small entity 226, the dictionary generator 126 may remove sets of the small entity 226 that are completely contained within the fragment 223. To illustrate this, index numbers for atoms in the entry for the small entity 226 that are contained within a larger substructure (fragment 223) are crossed out in FIG. 2B.

Referring again to FIG. 1, the distance calculator 129 generates a distance matrix for the target molecule 115 based on the atom indexes in the dictionary entries. Topological distances (“distances”) between pairs of atoms can be found using the distance matrix. Herein, “distance” refers to the shortest path (number of bonds) between two atoms. In order to determine a distance between a first and second substructure, distances between pairs of atoms can be determined from the distance matrix. Each pair can include an atom from the first substructure and an atom from the second substructure. In some embodiments, the distances between pairs of atoms within a given number of bond lengths (e.g., 2-6 bonds) of one another can be used to calculate a distance between the two substructures. The distance calculator 129 can calculate the average of these distances and the inverse or inverse square of each average. A topological distance for the two substructures can then be selected from these values (e.g., a minimum inverse square of average distances). A molecular descriptor 135 with this distance can then be generated and added to a predictive model 140.

FIG. 2C is a schematic diagram illustrating an example 203 wherein topological distances between substructures 210 are determined, according to some embodiments of the present disclosure. The topological distances between substructures 210 extracted and indexed in examples 201 and 202 (FIGS. 2A and 2B) can be found. FIG. 2C illustrates two of these substructures: fragment 243 and fragment 223. Sets of atom indexes in two substructure entries (a) and (b) (for fragment 243 and fragment 223, respectively) are compared. These atoms are indexed with fragment 243 in a first set (a-set1) and fragment 223 in first and second sets (b-set1 and b-set2).

The distances between pairs of atoms from entries (a) and (b) can be found in a distance matrix 250. The inverse squares of the averages of the a-set1/b-set1 distances and the a-set1/b-set2 distances can then be found (FIG. 2C, III). The minimum inverse square (III) can be selected as the topological distance between fragment 243 and fragment 223 (FIG. 2C (IV)). As shown in FIG. 1, this distance can be stored as a molecular descriptor 135 in a set of training data for a predictive model 140. Topological distances may be determined for at least one additional pair of the substructures 210 and added to the predictive model as well.

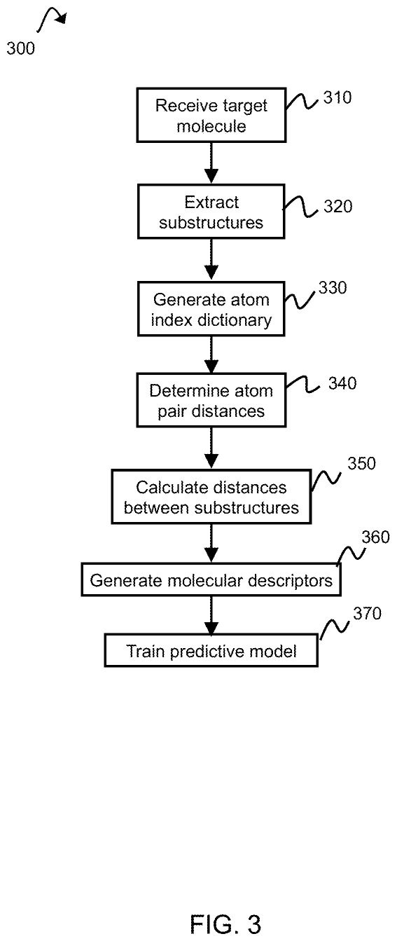

FIG. 3 is a flow diagram illustrating a process 300 of generating a predictive model, according to some embodiments of the present disclosure. To illustrate process 300, but not to limit embodiments, FIG. 3 is described within the context of environment 100 illustrated in FIG. 1. Where elements referred to in FIG. 3 are identical to elements shown in FIG. 1, the same reference numbers are used in each figure.

A set of training data can be received. This is illustrated at operation 310. The training data can include a set of molecules such as those discussed above with respect to FIG. 1 (e.g., target molecule 115). A set of substructures can be extracted (e.g., identified, generated) for each molecule. This is illustrated at operation 320. The molecules used to generate the molecular descriptors can have two or more substructures, which can include heteroatoms (i), fragments (ii), and/or small entities (iii). This is discussed in greater detail with respect to FIGS. 1 and 2A.

An atom index dictionary is generated for the substructures. This is illustrated at operation 330. The atom indexes and corresponding substructures can be stored in dictionary entries. In some embodiments, small entities (iii) completely contained within another substructure can be identified and removed at operation 330. Generating a dictionary for substructures and atom indexes is discussed in greater detail with respect to FIGS. 1 and 2B.

Distances between pairs of atoms in the dictionary can be determined. This is illustrated at operation 340. A distance matrix with the atom indexes can be used to determine these distances. An example of a distance matrix 250 that may be used is illustrated in FIG. 2C.

Topological distances between substructures are calculated based on the atom pair distances. This is illustrated at operation 350. The topological distances can be calculated using average distances between pairs of atoms from different substructures (“substructure atom pairs”). The substructures can include at least two substructures independently selected from heteroatoms, fragments, and small entities. Molecular descriptors are generated based on these distances. This is illustrated at operation 360. Molecular descriptors can be generated and stored using various values determined from the distances between substructures. These values may include minimum distance between two atoms of a substructure pair, an average distance between atoms of selected substructure atom pairs, an inverse of this average, an inverse square of this average, or any other appropriate restriction.

For example, molecular descriptors can be generated by calculating the distance between atoms of a substructure atom pair with a minimum number of bonds (“bond number”) between them. In some embodiments, an average value of distances between multiple substructure atom pairs with minimum bond numbers (e.g., more than one substructure atom pair separated by one bond) can be found, and the inverse square of this average of may be a molecular descriptor. The average distance can also be found for substructure atom pairs within a specified number of bonds (e.g., atoms separated by three bonds), and the inverse square of this average may be a molecular descriptor. This is discussed in greater detail with respect to FIGS. 1 and 2C.

The topological distance(s) between substructures are added to a predictive model 140. This is illustrated at operation 370. The predictive model 140 can be a model for predicting any appropriate properties that may be associated with the target molecule 115. For example, the model 140 can be a quantitative structure-activity relationship (QSAR) model. In experimental examples discussed below, robustness of the disclosed model 140 was tested using highest molecular orbital (HOMO) energy as a target property. The training data in these examples included 42 structures of organic compounds with molecular weights greater than about 550 Da and HOMO energies in a range of approximately −5.44 eV to −4.56 eV.

Ridge, lasso, and elastic net regression models were used to test model robustness. The expected accuracy was computed using a 5-fold-within-5-fold nested cross validation procedure. Hyperparameter tuning was carried out using different alpha values (α=1.0e−06, 1.0e−05, 1.0e−04, 1.0e−03, 1.0e−02, 1.0e−01, 1.0e+00, 1.0e+01, 1.0e+2, 1.0e+4) and different l1_ratio values (0.0, 0.2, 0.4, 0.6, 0.8, 1.0).

In a first of the experimental examples, molecular descriptors were generated based on distances between substructures including heteroatoms, fragments (extracted using BRICS), and small entities (extracted using Morgan fingerprint). The molecular descriptors were generated using topological distances between the substructures calculated by adding the inverse squares of average distances between substructure atom pairs within three bonds. The disclosed model 140 was trained on molecular descriptors generated from these distances and used to predict HOMO energy values.

In the first example, the robustness of the model 140 was compared to two conventional models: Morgan fingerprint (radius=1, 2) and atom pair fingerprint. The Morgan fingerprint molecular descriptors were based on counting a number of substructures (e.g., small entities). The atom pair fingerprint molecular descriptors were based on distances between pairs of atoms. The disclosed model 140 achieved higher cross-validation scores (R²) than Morgan fingerprint and atom pair fingerprint when the three models were used to target the aforementioned HOMO energies. Cross-validation scores determined by a ridge regression model were 0.59±0.15 (Morgan fingerprint), 0.12±0.30 (atom pair fingerprint), and 0.74±0.15 (disclosed model 140). Cross-validation scores determined by a lasso regression model were 0.56±0.10 (Morgan fingerprint), 0.27±0.25 (atom pair fingerprint), and 0.75±0.11 (disclosed model 140). Cross-validation scores (R²) determined by an elastic net regression model were 0.59±0.08 (Morgan fingerprint), 0.10±0.20 (atom pair fingerprint), and 0.74±0.08 (disclosed model 140).

Cross validation (ridge, lasso, and elastic net regression models) was also used to compare predictions of the disclosed model 140 with molecular descriptors generated based on different types of distance information. In this experimental example, each molecular descriptor was based on distances for heteroatom, fragment, and small entities (substructures). Types of distance information used to generate molecular descriptors are discussed in greater detail with respect to operation 340 of process 300. As in the previous example, cross-validation scores were determined for predictions based on these molecular descriptors using the model 140 generated at operation 350.

A first prediction used molecular descriptors generated based on distances between, for each pair of substructures, a substructure atom pair with a minimum bond number between them. The cross-validation scores for the first prediction were 0.72±0.15 (ridge), 0.70±0.18 (lasso), and 0.68±0.15 (elastic net). A second prediction used molecular descriptors generated based on, for each pair of substructures, inverse squares of the average distance between substructure atom pairs with a minimum bond number between them. The cross-validation scores for the second prediction were 0.74±0.16 (ridge), 0.66±0.19 (lasso), and 0.69±0.15 (elastic net). A third prediction used molecular descriptors generated based on, for each pair of substructures, a sum of the inverse squares of average distances between substructure atom pairs within three bond numbers. The cross-validation scores for the third prediction were 0.74±0.15 (ridge), 0.75±0.11 (lasso), and 0.74±0.08 (elastic net).

In another experimental example, cross validation (ridge, lasso, and elastic net regression models) was used to compare predictions of the disclosed model 140 with molecular descriptors generated using different types of substructures. Each molecular descriptor was based on a sum of the inverse squares of average distances between substructure atom pairs within three bond numbers, as in the third prediction tested in the above example. Types of substructures that can be used to generate molecular descriptors are discussed in greater detail with respect to operation 330 of process 300. As in the previous examples, cross-validation scores were determined for predictions based on these molecular descriptors using the model 140 generated at operation 350.

In this example, a prediction was made using molecular descriptors generated based on distances between heteroatom and small entity substructures. That is, molecular fragments were not included in the sets of substructures generated at operation 330 when making this prediction. The cross-validation scores for this prediction were 0.69±0.13 (ridge), 0.65±0.16 (lasso), and 0.65±0.11 (elastic net). It is noted that these scores were lower than those of the aforementioned (third) prediction, which used molecular descriptors generated based on sums of inverse squares of average distances between heteroatom, fragment, and small entity substructure atom pairs within three bond numbers.

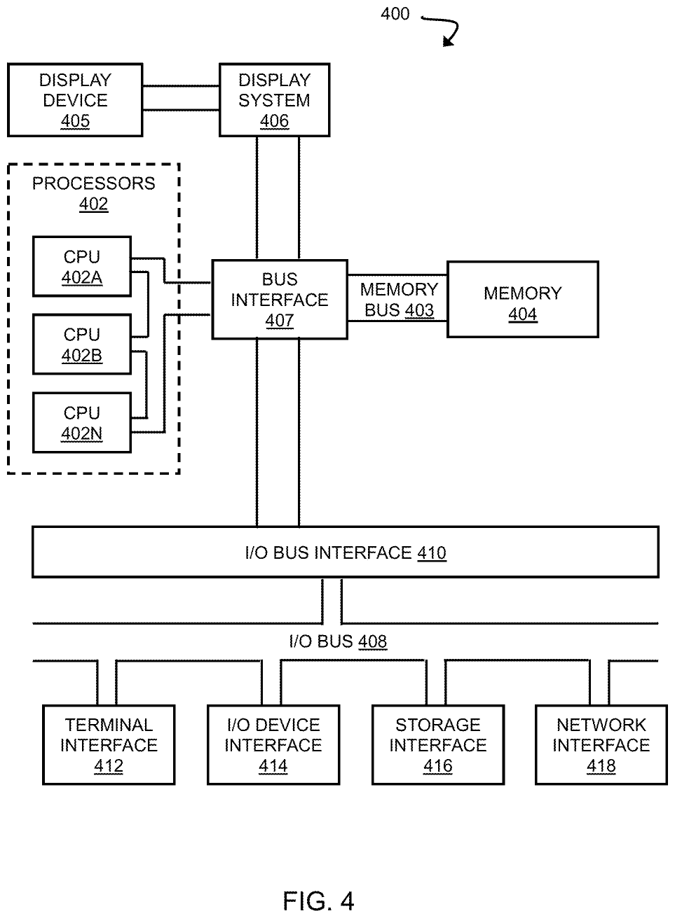

FIG. 4 is a block diagram illustrating an exemplary computer system 400 that can be used in implementing one or more of the methods, tools, components, and any related functions described herein (e.g., using one or more processor circuits or computer processors of the computer). In some embodiments, the major components of the computer system 400 comprise one or more processors 402, a memory subsystem 404, a terminal interface 412, a storage interface 416, an input/output device interface 414, and a network interface 418, all of which can be communicatively coupled, directly or indirectly, for inter-component communication via a memory bus 403, an input/output bus 408, bus interface unit 407, and an input/output bus interface unit 410.

The computer system 400 contains one or more general-purpose programmable central processing units (CPUs) 402A, 402B, and 402-N, herein collectively referred to as the CPU 402. In some embodiments, the computer system 400 contains multiple processors typical of a relatively large system; however, in other embodiments the computer system 400 can alternatively be a single CPU system. Each CPU 402 may execute instructions stored in the memory subsystem 404 and can include one or more levels of on-board cache.

The memory 404 can include a random-access semiconductor memory, storage device, or storage medium (either volatile or non-volatile) for storing or encoding data and programs. In some embodiments, the memory 404 represents the entire virtual memory of the computer system 400 and may also include the virtual memory of other computer systems coupled to the computer system 400 or connected via a network. The memory 404 is conceptually a single monolithic entity, but in other embodiments the memory 404 is a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory can be further distributed and associated with different CPUs or sets of CPUs, as is known in any of various so-called non-uniform memory access (NUMA) computer architectures.

Components of environment 100 (FIG. 1) can be included within the memory 404 in the computer system 400. However, in other embodiments, some or all of these components may be on different computer systems and may be accessed remotely, e.g., via a network. The computer system 400 may use virtual addressing mechanisms that allow the programs of the computer system 400 to behave as if they only have access to a large, single storage entity instead of access to multiple, smaller storage entities. Thus, components of the memory 404 are not necessarily all completely contained in the same storage device at the same time. Further, although components of environment 100 are illustrated as being separate entities, in other embodiments some of these components, portions of some of these components, or all of these components may be packaged together.

In an embodiment, components of environment 100 include instructions that execute on the processor 402 or instructions that are interpreted by instructions that execute on the processor 402 to carry out the functions as further described in this disclosure. In another embodiment, components of environment 100 are implemented in hardware via semiconductor devices, chips, logical gates, circuits, circuit cards, and/or other physical hardware devices in lieu of, or in addition to, a processor-based system. In another embodiment, components of environment 100 include data in addition to instructions.

Although the memory bus 403 is shown in FIG. 4 as a single bus structure providing a direct communication path among the CPUs 402, the memory subsystem 404, the display system 406, the bus interface 407, and the input/output bus interface 410, the memory bus 403 can, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the input/output bus interface 410 and the input/output bus 408 are shown as single respective units, the computer system 400 may, in some embodiments, contain multiple input/output bus interface units 410, multiple input/output buses 408, or both. Further, while multiple input/output interface units are shown, which separate the input/output bus 408 from various communications paths running to the various input/output devices, in other embodiments some or all of the input/output devices may be connected directly to one or more system input/output buses.

The computer system 400 may include a bus interface unit 407 to handle communications among the processor 402, the memory 404, a display system 406, and the input/output bus interface unit 410. The input/output bus interface unit 410 may be coupled with the input/output bus 408 for transferring data to and from the various input/output units. The input/output bus interface unit 410 communicates with multiple input/output interface units 412, 414, 416, and 418, which are also known as input/output processors (IOPs) or input/output adapters (IOAs), through the input/output bus 408. The display system 406 may include a display controller. The display controller may provide visual, audio, or both types of data to a display device 405. The display system 406 may be coupled with a display device 405, such as a standalone display screen, computer monitor, television, or a tablet or handheld device display. In alternate embodiments, one or more of the functions provided by the display system 406 may be on board a processor 402 integrated circuit. In addition, one or more of the functions provided by the bus interface unit 407 may be on board a processor 402 integrated circuit.

In some embodiments, the computer system 400 is a multi-user mainframe computer system, a single-user system, or a server computer or similar device that has little or no direct user interface but receives requests from other computer systems (clients). Further, in some embodiments, the computer system 400 is implemented as a desktop computer, portable computer, laptop or notebook computer, tablet computer, pocket computer, telephone, smart phone, network switches or routers, or any other appropriate type of electronic device.

It is noted that FIG. 4 is intended to depict the representative major components of an exemplary computer system 400. In some embodiments, however, individual components may have greater or lesser complexity than as represented in FIG. 4, Components other than or in addition to those shown in FIG. 4 may be present, and the number, type, and configuration of such components may vary.

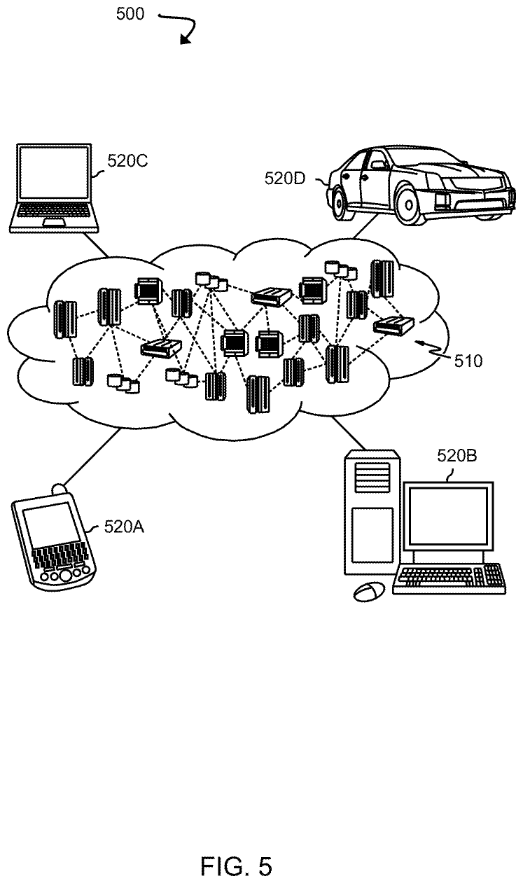

In some embodiments, the data storage and retrieval processes described herein could be implemented in a cloud computing environment, which is described below with respect to FIGS. 4 and 5. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher-level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 5 is a block diagram illustrating a cloud computing environment 500, according to some embodiments of the present disclosure. As shown, cloud computing environment 500 includes one or more cloud computing nodes 510 with which local computing devices used by cloud consumers, such as, for example, personal digital assistant (PDA) or cellular telephone 520A, desktop computer 520B, laptop computer 520C, and/or automobile computer system 520D may communicate. Nodes 510 may communicate with one another. They may be grouped (not shown) physically or virtually, in one or more networks, such as Private, Community, Public, or Hybrid clouds as described hereinabove, or a combination thereof. This allows cloud computing environment 500 to offer infrastructure, platforms and/or software as services for which a cloud consumer does not need to maintain resources on a local computing device. It is understood that the types of computing devices 520A—520D shown in FIG. 4 are intended to be illustrative only and that computing nodes 510 and cloud computing environment 500 can communicate with any type of computerized device over any type of network and/or network addressable connection (e.g., using a web browser).

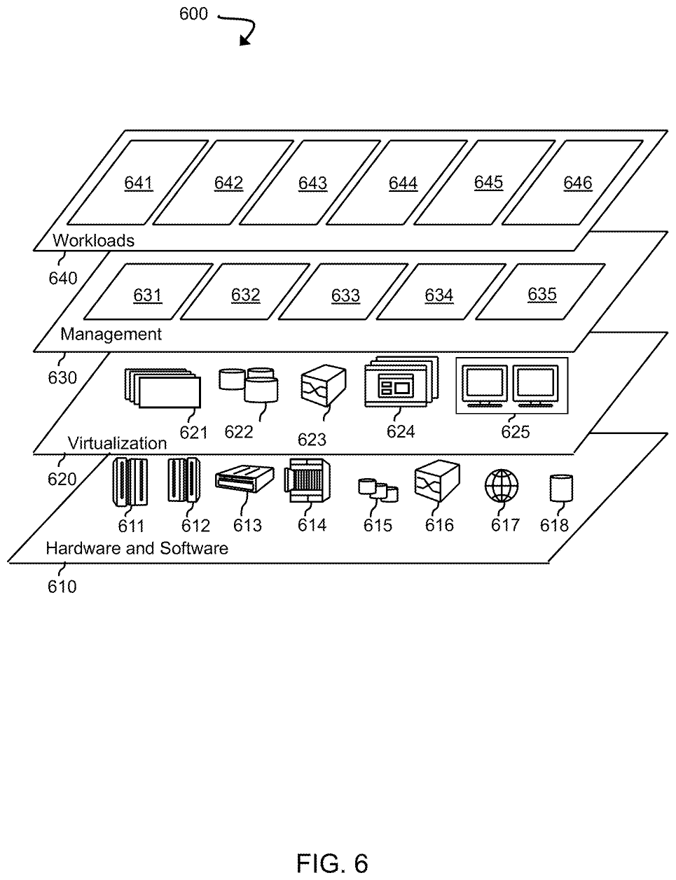

FIG. 6 is a block diagram illustrating a set of functional abstraction model layers 600 provided by the cloud computing environment 500, according to some embodiments of the present disclosure. It should be understood in advance that the components, layers, and functions shown in FIG. 6 are intended to be illustrative only and embodiments of the invention are not limited thereto. As depicted, the following layers and corresponding functions are provided:

Hardware and software layer 610 includes hardware and software components. Examples of hardware components include: mainframes 611; RISC (Reduced Instruction Set Computer) architecture-based servers 612; servers 613; blade servers 614; storage devices 615; and networks and networking components 616. In some embodiments, software components include network application server software 617 and database software 618.

Virtualization layer 620 provides an abstraction layer from which the following examples of virtual entities may be provided: virtual servers 621; virtual storage 622; virtual networks 623, including virtual private networks; virtual applications and operating systems 624; and virtual clients 625.

In one example, management layer 630 provides the functions described below. Resource provisioning 631 provides dynamic procurement of computing resources and other resources that are utilized to perform tasks within the cloud computing environment. Metering and Pricing 632 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 633 provides access to the cloud computing environment for consumers and system administrators. Service level management 634 provides cloud computing resource allocation and management such that required service levels are met. Service Level Agreement (SLA) planning and fulfillment 635 provide pre-arrangement for, and procurement of, cloud computing resources for which a future requirement is anticipated in accordance with an SLA.

Workloads layer 640 provides examples of functionality for which the cloud computing environment can be utilized. Examples of workloads and functions that can be provided from this layer include: mapping and navigation 641; software development and lifecycle management 642; virtual classroom education delivery 643; data analytics processing 644; transaction processing 645; and molecular descriptor generating 646.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a standalone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the present disclosure.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof.

In the previous detailed description of example embodiments of the various embodiments, reference was made to the accompanying drawings (where like numbers represent like elements), which form a part hereof, and in which is shown by way of illustration specific example embodiments in which the various embodiments may be practiced. These embodiments were described in sufficient detail to enable those skilled in the art to practice the embodiments, but other embodiments may be used and logical, mechanical, electrical, and other changes may be made without departing from the scope of the various embodiments. In the previous description, numerous specific details were set forth to provide a thorough understanding the various embodiments. But, the various embodiments may be practiced without these specific details. In other instances, well-known circuits, structures, and techniques have not been shown in detail in order not to obscure embodiments.

When different reference numbers comprise a common number followed by differing letters (e.g., 100a, 100b, 100c) or punctuation followed by differing numbers (e.g., 100-1, 100-2, or 100.1, 100.2), use of the reference character only without the letter or following numbers (e.g., 100) may refer to the group of elements as a whole, any subset of the group, or an example specimen of the group.

As used herein, “a number of” when used with reference to items, means one or more items. For example, “a number of different types of networks” is one or more different types of networks.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, and item C” may include item A, item A and item B, or item B. This example also may include item A, item B, and item C or item B and item C. Of course, any combinations of these items can be present. In some illustrative examples, “at least one of” can be, for example, without limitation, two of item A; one of item B; ten of item C; four of item B and seven of item C; or other suitable combinations.

What is claimed is:

1. A method, comprising:
receiving a target molecule;
extracting substructures from the target molecule;
generating a distance matrix for a pair of the substructures based on corresponding atom indexes;
determining distances between atoms of the pair of the substructures based on the distance matrix;
calculating a distance between the pair of the substructures based on at least one of the distances between the atoms of the pair of the substructures; and
generating a molecular descriptor based on the distance between the pair of the substructures.

2. The method of claim 1, wherein the substructures are independently selected from the group consisting of molecular fragments, small entities, and heteroatoms.

3. The method of claim 2, wherein the small entities comprise at least three non-hydrogen atoms.

4. The method of claim 1, wherein the at least one of the distances between the atoms comprises a distance between atoms within a given number of bonds.

5. The method of claim 4, further comprising,
when more than two atoms from the pair of the substructures are within the given number of bonds, calculating an average distance between the more than two atoms; and
calculating an inverse square of the average distance.

6. The method of claim 4, wherein the given number of bonds is six bonds.

7. The method of claim 1, further comprising generating a molecular descriptor based on distances between more than one pair of the substructures.

8. A system, comprising:
a memory; and
a processor communicatively coupled to the memory, wherein the processor is configured to perform a method comprising:
receiving a target molecule;
extracting substructures from the target molecule;
generating a distance matrix for a pair of the substructures based on corresponding atom indexes;
determining distances between atoms of the pair of the substructures based on the distance matrix;
calculating a distance between the pair of the substructures based on at least one of the distances between the atoms of the pair of the substructures; and
generating a molecular descriptor based on the distance between the pair of the substructures.

9. The system of claim 8, wherein the substructures are independently selected from the group consisting of molecular fragments, small entities, and heteroatoms.

10. The system of claim 9, wherein the small entities comprise at least three non-hydrogen atoms.

11. The system of claim 8, wherein the at least one of the distances between the atoms comprises a distance between atoms within a given number of bonds.

12. The system of claim 11, wherein the method further comprises:
when more than two atoms from the pair of the substructures are within the given number of bonds, calculating an average distance between the more than two atoms; and
calculating an inverse square of the average distance.

13. The system of claim 11, wherein the given number of bonds is six bonds.

14. The system of claim 1, wherein the method further comprises generating a molecular descriptor based on distances between more than one pair of the substructures.

15. A computer program product, the computer program product comprising a computer readable storage medium having program instructions embodied therewith, the program instructions executable by a processor to cause a device to perform a method, the method comprising:
receiving a target molecule;
extracting substructures from the target molecule;
generating a distance matrix for a pair of the substructures based on corresponding atom indexes;
determining distances between atoms of the pair of the substructures based on the distance matrix;
calculating a distance between the pair of the substructures based on at least one of the distances between the atoms of the pair of the substructures; and
generating a molecular descriptor based on the distance between the pair of the substructures.

16. The computer program product of claim 15, wherein the substructures are independently selected from the group consisting of molecular fragments, small entities, and heteroatoms.

17. The computer program product of claim 15, wherein the at least one of the distances between the atoms comprises a distance between atoms within a given number of bonds.

18. The computer program product of claim 17, wherein the method further comprises:
when more than two atoms from the pair of the substructures are within the given number of bonds, calculating an average distance between the more than two atoms; and
calculating an inverse square of the average distance.

19. The system of claim 17, wherein the given number of bonds is six bonds.

20. The system of claim 15, wherein the method further comprises generating a molecular descriptor based on distances between more than one pair of the substructures.
